(19) United States
(12) Patent Application Publication
(10) Pub. No.: US 2009/0301480 A1
(43) Pub. Date: Dec. 10, 2009

(54) DIAGNOSTIC SAMPLE COLLECTION SYSTEM AND METHOD OF USE

(76) Inventors: Mamdouh Elsakka, Corona, CA (US); Christine T. Kearney, Glen Ellyn, IL (US); Ho-Kin Ng, Corona, CA (US); John Edrington, Jeffersonville, IN (US); Keith Wells, La Grange, KY (US); Greg Furnish, Louisville, KY (US); Eric E. Bielefeld, New Albany, IN (US)

Correspondence Address: DICKE, BILLIG & CZAJA, PLLC;ATTN: CFN MATTERS, 100 SOUTH FIFTH STREET, SUITE 2250, MINNEAPOLIS, MN, 55402, US

(21) Appl. No.: 12/135,880

(22) Filed: Jun. 9, 2008

(51) Int. Cl. A61M 16/00

(52) U.S. Cl. 128/202.16

(57) ABSTRACT

A bronchoalveolar lavage instillation/aspiration system which includes (i) a needleless bronchoalveolar lavage instillation/aspiration device with a barrel member including a barrel lumen having a distal aperture and a self-sealing member configured for maintaining a fluid-disruptable, resealable barrier to the distal aperture; and (ii) a catheter assembly configured to provide a patent path of fluid communication between the instillation/aspiration device and a patient bronchial passage, where the catheter includes a wedging structure configured to engage a patient bronchial passage. In another aspect, embodiments of the present invention may include one or more methods of making and using a system or component described herein.

### TECHNICAL FIELD

Embodiments of the present invention relate generally to medical devices, and more particularly to a system for sample collection in a non-bronchoscopic bronchoalveolar lavage procedure.

### BACKGROUND

Non-bronchoscopic bronchoalveolar lavage (BAL) is a medical procedure commonly used for diagnoses related to infections (such as, for example, ventilator-associated pneumonia (VAP)) in patients under mechanical ventilation or otherwise at risk for lung infections such as those with depressed immune systems, lung cancer, or interstitial lung disease. VAP is a general term encompassing pneumonia-associated infections for which patients under mechanical ventilation (i.e., connected to a respirator/ventilator or similar mechanical device for assisting respiration) are at higher risk, with specific reference to hospital-acquired infections. A non-bronchoscopic BAL procedure, described with reference to FIG. 1 is performed on an intubated patient 105 who is on a mechanical ventilator (diagrammatically shown as 107). A respiratory technician (not shown) directs a catheter 101 through a manifold 103 connected to the patient's endotracheal tube 109. The distal end 111 of the catheter 101 is guided through the trachea 113 and into a lower region of a lung 112 where its quoit-shaped polymer tip 111 is “wedged” into a bronchial passage 115 to form a discrete volume area that is generally isolated from the surrounding lung (which is shown magnified in FIG. 1A). A syringe 117 is connected to the proximal end of the catheter 101 and used to introduce a volume of sterile saline solution into the area via the catheter 101. The fluid is then withdrawn, together with any material from that area, which may include proteins and microorganisms (if present), collectively referred to as “fluid sample.” Next, the respiratory technician transfers the fluid sample from the syringe 117 to a sputum cup 119, which is sealed and sent to a diagnostic laboratory for testing to determine whether there are any cytological or microbial issues that need addressed for the patient.

Bronchoscopic bronchoalveolar lavage has long been known in the art. Non-bronchoscopic alveolar lavage provides advantages over the bronchoscopic procedure including lower cost due to the removal of need for expensive bronchoscopy equipment, the ability for a respiratory technician to conduct the procedure rather than a physician, and the ability to use disposable components to reduce the costs and potential risks associated with sterilization and re-use of bronchoscopic equipment. The bronchoscopic procedure commonly requires the patient to be sedated, which adds expense and poses an increased risk for patients that often have other respiratory complications.

However, even with the advent of non-bronchoscopic BAL procedures, there is still a need for improved equipment that will promote patient safety, comfort, and economy. In particular, there is a need for non-bronchoscopic BAL equipment that will provide a reduced risk of transporting contaminants from the upper respiratory tract into the lower lung. There is also a need to decrease the likelihood that a fluid sample will be contaminated between collection and diagnostic analysis. And, there is also a need to increase the efficiency with which non-bronchoscopic BAL equipment may be used to decrease the time that a procedure takes a caregiver to perform, as well as the time during which a patient must endure the discomfort of an invasive device in his lower lung.

### BRIEF SUMMARY

In one aspect, embodiments of the present invention may include a system for non-bronchoscopic bronchoalveolar lavage including a self-contained assembly for installation and aspiration of fluid and a catheter assembly. In another aspect, embodiments of the present invention may include a catheter assembly including an inner catheter member with a distal wedging means and an outer catheter including a disruptably sealed distal end configured to allow disruption by and passage of the inner catheter member. In yet another aspect, embodiments of the present invention may include a self-contained assembly for installation and aspiration of fluid including a distal self-sealing component and being dimensioned for use as a standard cytological sputum cup.

In still another aspect, embodiments of the present invention may include a bronchoalveolar lavage instillation/aspiration system which includes (i) a needleless bronchoalveolar lavage instillation/aspiration device with a barrel member including a barrel lumen having a distal aperture and a self-sealing member configured for maintaining a fluid-disruptable, resealable barrier to the distal aperture; and (ii) a catheter assembly configured to provide a patent path of fluid communication between the instillation/aspiration device and a patient bronchial passage, where the catheter includes a wedging structure configured to engage a patient bronchial passage. In another aspect, embodiments of the present invention may include one or more methods of making and using a system or component described herein.

### BRIEF DESCRIPTION OF THE DRAWINGS

FIG. 1 is a diagrammatic illustration of a non-bronchoscopic bronchoalveolar lavage procedure;

FIG. 1A is a diagrammatic illustration of a lung portion with a wedging catheter therein;

FIG. 2 is a partially exploded diagram of a non-bronchoscopic bronchoalveolar lavage system embodiment;

FIG. 3A is a disassembled instillation/aspiration assembly embodiment;

FIG. 3B is an illustration of the instillation/aspiration assembly embodiment of FIG. 3A, assembled;

FIG. 4A is an embodiment of another instillation/aspiration assembly embodiment with an off-center distal sealing member;

FIG. 4B is an embodiment of yet another instillation/aspiration assembly embodiment with a generally centered distal sealing member;

FIG. 4C is an embodiment of still another instillation/aspiration assembly embodiment;

FIG. 5 is an exploded view of a manifold assembly embodiment;

FIGS. 6A-6G are views of components of a twist-lock member of the manifold assembly of FIG. 5;

FIGS. 7A-7C are, respectively, distal perspective, proximal perspective, and longitudinal section views of a dual-diaphragm member of the manifold assembly of FIG. 5;

FIG. 8 is an exploded view of a catheter assembly;

FIG. 8A is a detail view of a distal inner catheter end including a wedging member of the catheter assembly of FIG. 8;

FIG. 8B is a perspective view of a squeeze-lock component of the catheter assembly of FIG. 8;

FIGS. 8C and 8D are, respectively, a side view and end perspective view of a distal portion of an outer catheter including a disruptable seal of the catheter assembly of FIG. 8;

FIGS. 8E and 8F are, respectively, a perspective view and a side view of another squeeze-lock component embodiment;

FIGS. 9A-9D are views of another embodiment of an outer catheter including a disruptable seal;

FIG. 10 is a distal perspective view of another embodiment of a wedging member, shown deployed as a wedged catheter;

FIGS. 11A-11D are side views and longitudinal section views of another embodiment of a wedging member;

FIGS. 12A-12B show, respectively, a longitudinal section view and an applied perspective view of yet another embodiment of a wedging member; and

FIGS. 13A-13M shows a method of using a bronchoalveolar lavage system.

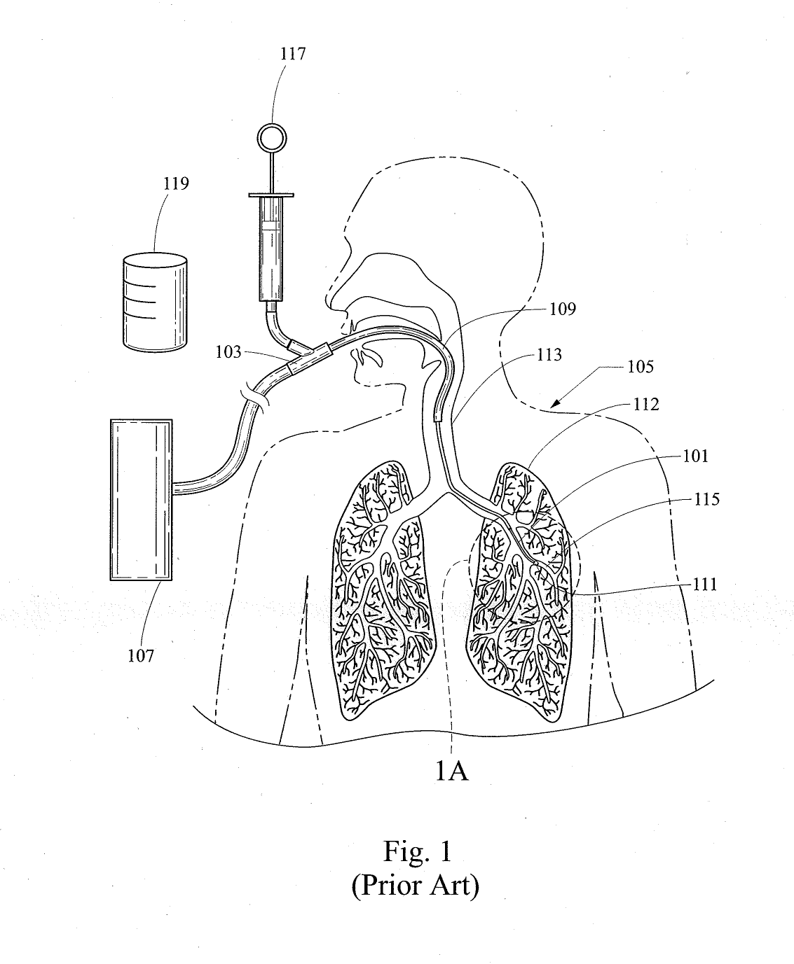

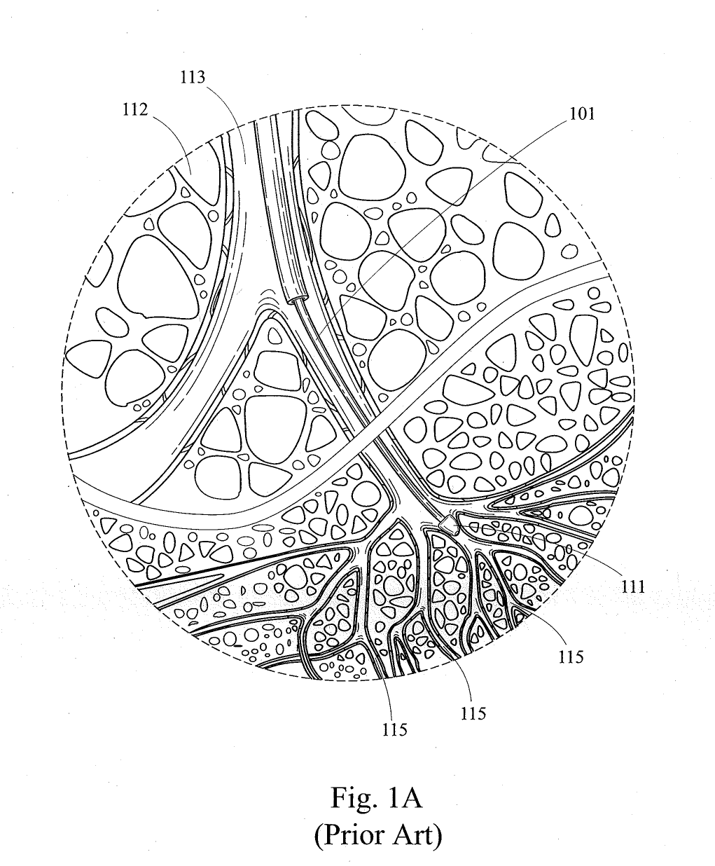

### DETAILED DESCRIPTION OF THE DRAWINGS AND CERTAIN EMBODIMENTS

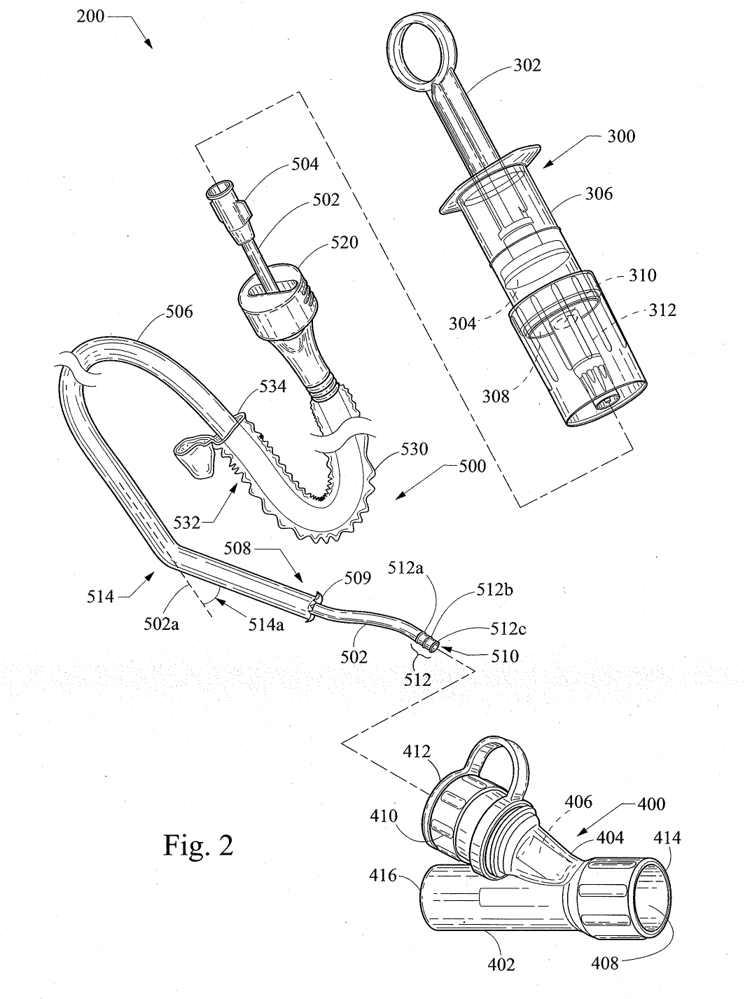

An embodiment of a non-bronchoscopic bronchoalveolar lavage system 200 is shown partially disassembled in FIG. 2. Other embodiments of each of its components are described in more detail below and may be used with a non-bronchoscopic bronchoalveolar lavage system of the present invention. The system 200 includes a self-contained assembly 300 for installation and aspiration of fluid, a manifold assembly 400, and a catheter assembly 500.

The instillation/aspiration assembly 300 is generally embodied as a syringe 300. The syringe 300 includes a handle 302 attached to a plunger 304 and a barrel 306, the interior portion of which includes a barrel lumen. The handle 302 is configured to actuate the plunger 304 distally and proximally along a central longitudinal axis of the barrel 306. Those of skill in the art will appreciate that distal-ward handle actuation will increase pressure in the barrel lumen, and proximal-ward actuation will decrease pressure in the barrel lumen, creating a partial vacuum therein. A distal wall 308 of the barrel 306 is generally transverse to its longitudinal axis and includes an aperture 310. A self-sealing member 312 extends distally from the aperture 310. In one embodiment, the self-sealing member 312 will provide a seal to the aperture 310 that is opened upon connection of the self-sealing member with a hub of a catheter device. Alternatively, the self-sealing member may be configured with a fluid-disruptable seal to the aperture 310 such that, at an ambient pressure, a seal is present that prevents fluid from passing through the self-sealing member 312. Then, when fluid subject to a predetermined pressure contacts the self-sealing member 312 (e.g., when the handle 302 and plunger 304 are actuated), the fluid will be able to pass through the self-sealing member. Those of skill in the art will appreciate that a number of different self-sealing mechanisms are known in the art that may be used within the scope of the present invention including, for example, the self-sealing mechanisms described in U.S. Pat. Nos. 5,405,333; 5,848,994; 6,206,860; 6,485,472; 6,745,998; 6,964,406; and 7,140,592 (each of which is assigned to a Cardinal Health company and is incorporated herein by reference in its entirety), Alaris® SmartSite® connectors or the like, technology described in U.S. Pat. Nos. 5,230,706; 5,360,413 or other self-sealing mechanisms particularly including bi-directional valve mechanisms. A generally cylindrical wall portion 314 extends distally beyond the main body of the barrel 306 and surrounds the self-sealing member 312. Some or all of the barrel 306 may be constructed of a transparent or translucent material to allow a user to view the contents of the barrel lumen.

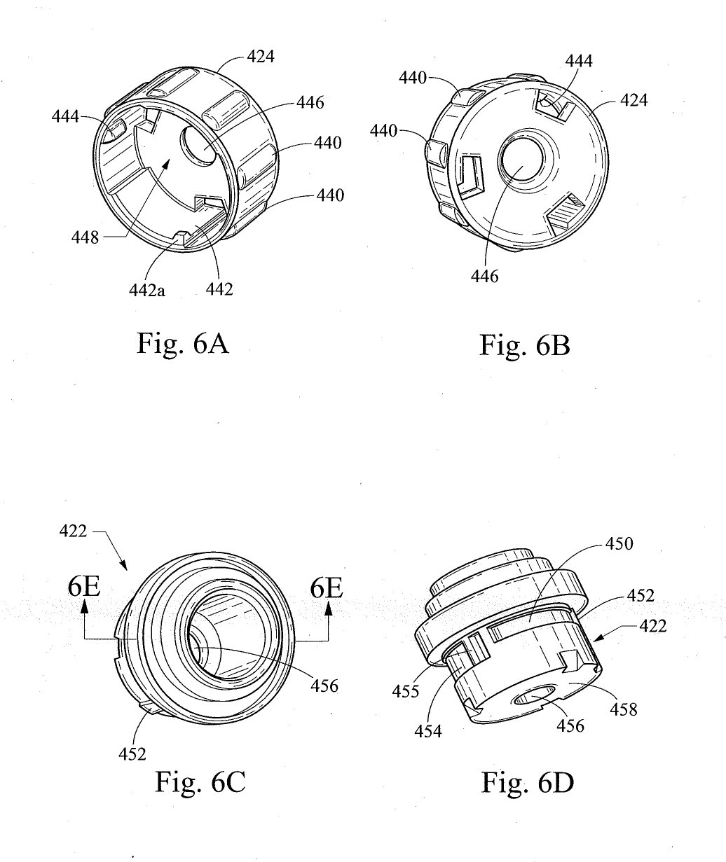

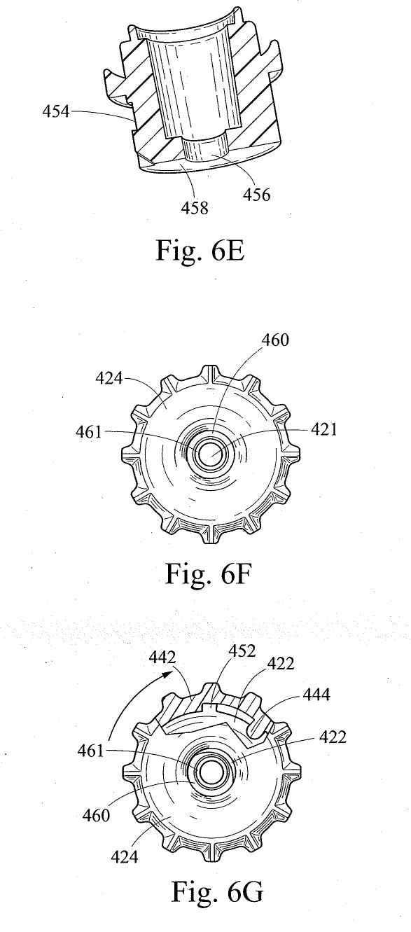

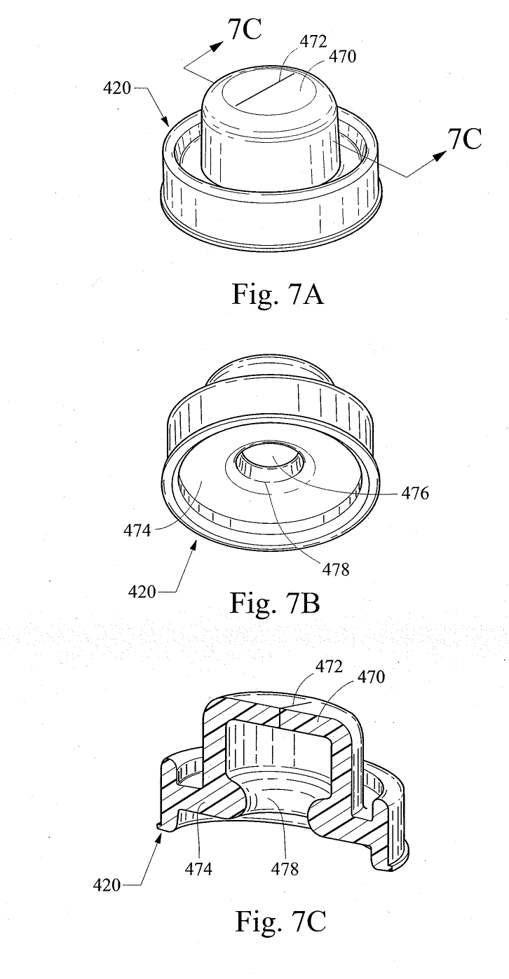

The manifold assembly 400 includes a generally tubular main body 402 and a generally tubular side branch 404 disposed at an angle to the main body 402. The side branch 404 includes a side branch lumen 406, which is continuous with a longitudinal main body lumen 408. The side branch lumen 406 encloses a dual-diaphragm seal (not shown, described below with reference to FIGS. 7A-7C). An upper portion of the side branch 404 includes a twist-lock mechanism 410 (described below with reference to FIGS. 6A-6G), which includes a central opening configured for passage therethrough of a portion of the catheter assembly 500. It also includes a cap member 412 for sealing that central opening. A distal end portion 414 of the main body 402 preferably is configured for connection to a patient's endotracheal tube, and a proximal end portion 416 preferably is configured for connection to the patient's circuit wye and/or closed suction catheter (not shown).

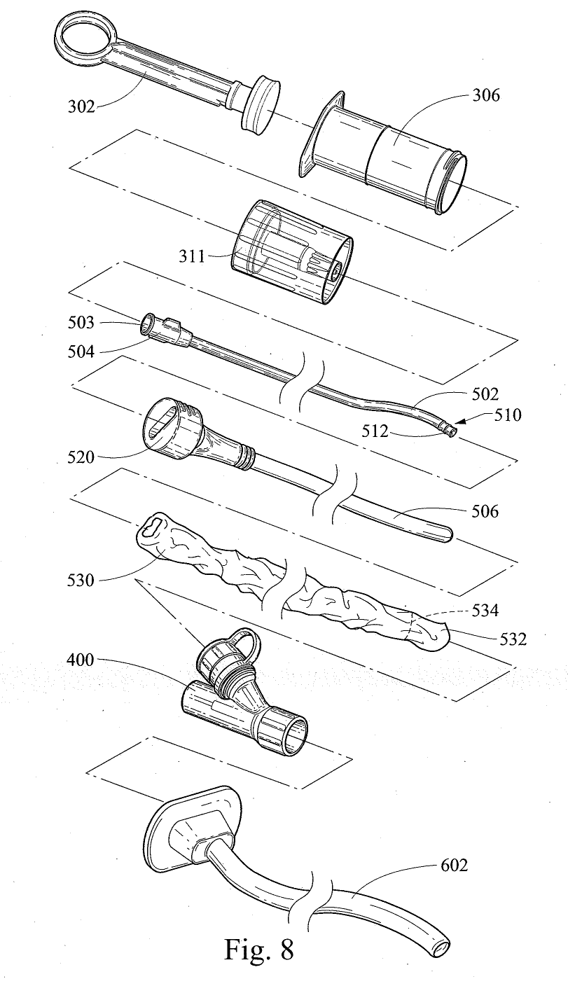

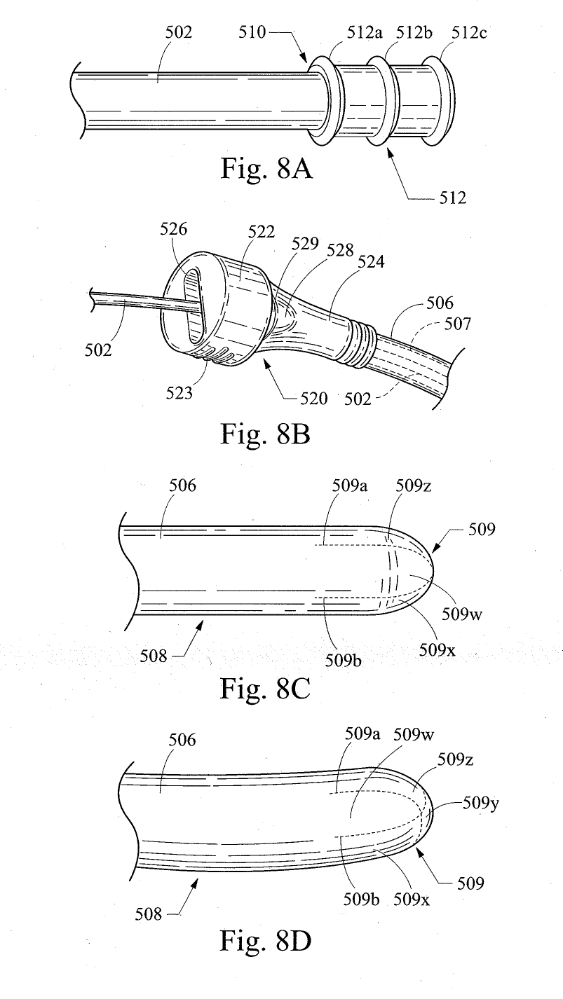

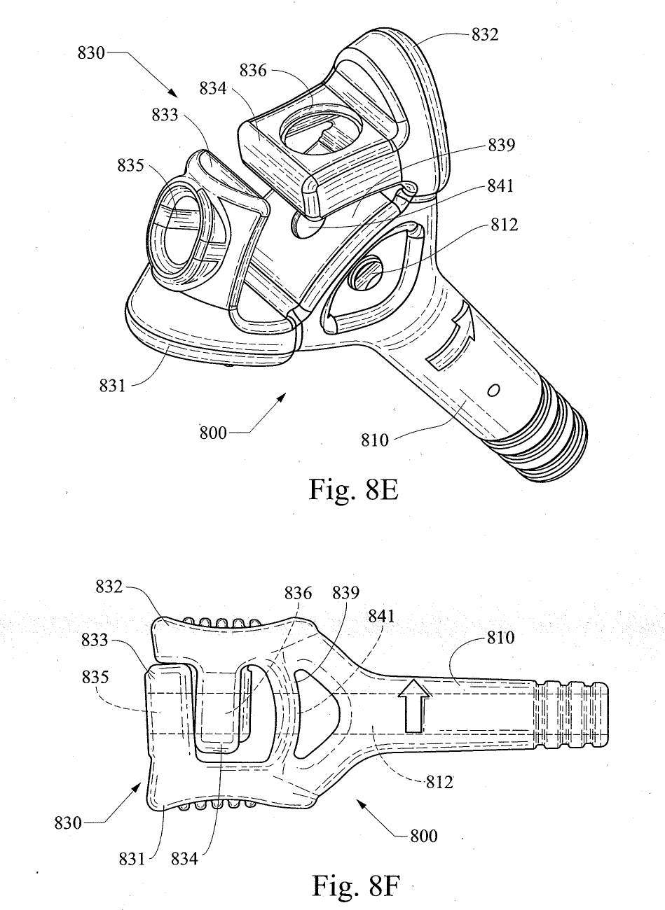

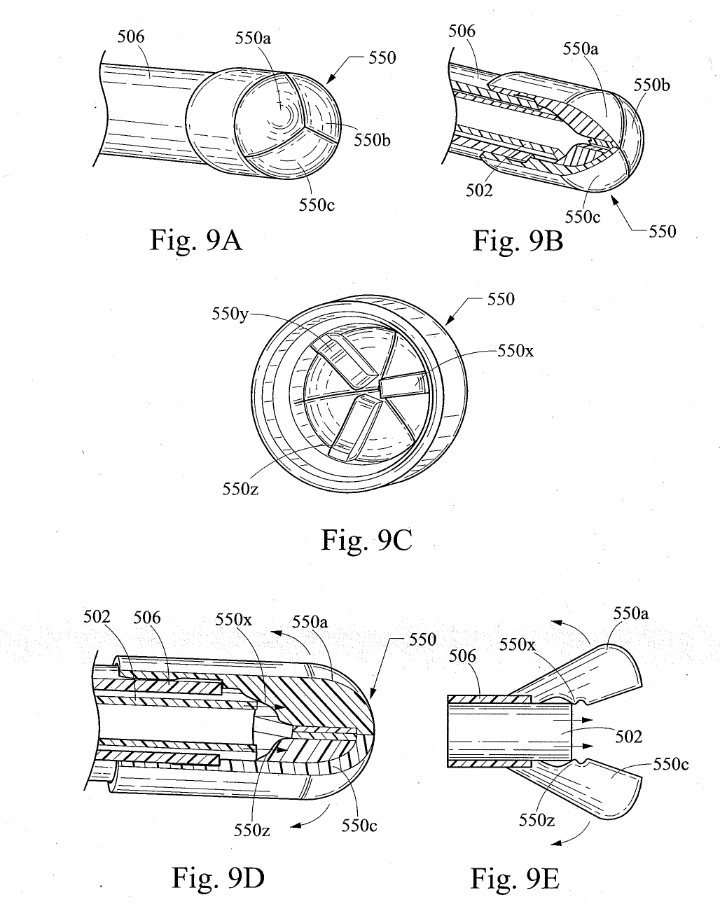

The catheter assembly 500 includes an inner catheter 502 with a proximal connection hub 504 configured for connection with the self-sealing member 312 in a manner that will provide a patent path of fluid communication between the barrel lumen and a lumen of the inner catheter. The inner catheter 502 is longitudinally and coaxially disposed through at least a lengthwise portion of a lumen of an outer catheter 506. In the illustrated embodiment, the inner catheter 502 is longer than the outer catheter 506. A distal end 508 of the outer catheter 506 includes an atraumatically-shaped disruptable seal 509, including a pair of overlapping slits that extend at least partially through an internal distal end wall portion of the outer catheter 506 (which structure is described below in greater detail with reference to different embodiments in FIGS. 8C-8D and 9A-9E). In various embodiments, there may be only a single slit, or there may be a plurality of overlapping slits that are on the same or different surfaces of the seal 509. As shown in FIG. 2, the end seal 509 has been disrupted by the distal end 510 of the inner catheter 502 having been forced through it.

The distal inner catheter end 510 includes a wedging structure 512 that is configured to at least partially sealingly contact an inner circumference of a passage in a lower portion of a patient lung (not shown; for purposes of the present application, the term “patient” may refer to a human or non-human animal that may be subjected to a bronchoalveolar lavage procedure). In this embodiment, the wedging structure 512 includes three adjacent flexible intact disc structures 512a-c disposed around an outer circumference of the inner catheter and generally transverse to its longitudinal axis. The outer catheter 506 preferably is constructed of a material of sufficient stiffness to retain a pre-formed bend 514 at a predetermined curve or angle 514a, but need not be rigid enough to allow 1:1 distal rotatability based upon rotation of a proximal portion. The preformed bend preferably is disposed at an angle selected to correspond within a typical angle range of a patient's tracheal-bronchial junction (i.e., at the inferior bifurcation of the trachea), and most preferably is configured to navigate that junction without colliding with the carina (i.e., as used herein, the phrase “preformed bend” includes a sharp bend, soft bend, arc, or any other shape whereby a portion of the outer catheter distal of the preformed bend is oriented out of the longitudinal axis of a portion proximal thereof). For example, in a system configured for use with a human patient may be set between about 10 and about 60 degrees, and preferably is set about 30 degrees out of the central longitudinal axis 502a of the inner catheter 502. This preformed bend 514 preferably is configured to allow a user to more easily direct the outer catheter from the patient's trachea into a desired bronchial branch while having only minimal contact with the walls of the patient's trachea and bronchi, without rotating the catheter after it is in the patient. For example, in a preferred embodiment, a user may introduce the catheter through the manifold at a predetermined rotational position that will correspond with entry of the distal catheter end into the desired patient lung (e.g., when it is desired to enter a patient's right lung, before introduction of the catheter, the manifold may be rotated such that the side branch through which the catheter will pass is oriented toward the patient's right side, then—when the catheter is directed through the side branch, the angled distal tip will also be oriented to point toward the patient's right side).

In the embodiment shown in FIG. 2, the catheter assembly 500 also includes a squeeze-lock component 520 and an outer sheath 530. The outer sheath 530 is configured to maintain a sterile or near-sterile condition of the covered portion of the catheter assembly 500. The distal end portion 532 of the outer sheath 530 preferably includes a perforated or other disruptable portion 534 (shown as disrupted/opened) allowing at least the distal end 532 to be opened for the outer catheter 506 to be passed through the twist-lock 410 of the manifold side branch 404. In other embodiments, substantially the entire length of the outer sheath may be removable. The squeeze-lock component 520, which is discussed below in greater detail with reference to FIG. 8B, is configured to releasably retain the longitudinal position of the inner catheter 502 relative to that of the outer catheter 506.

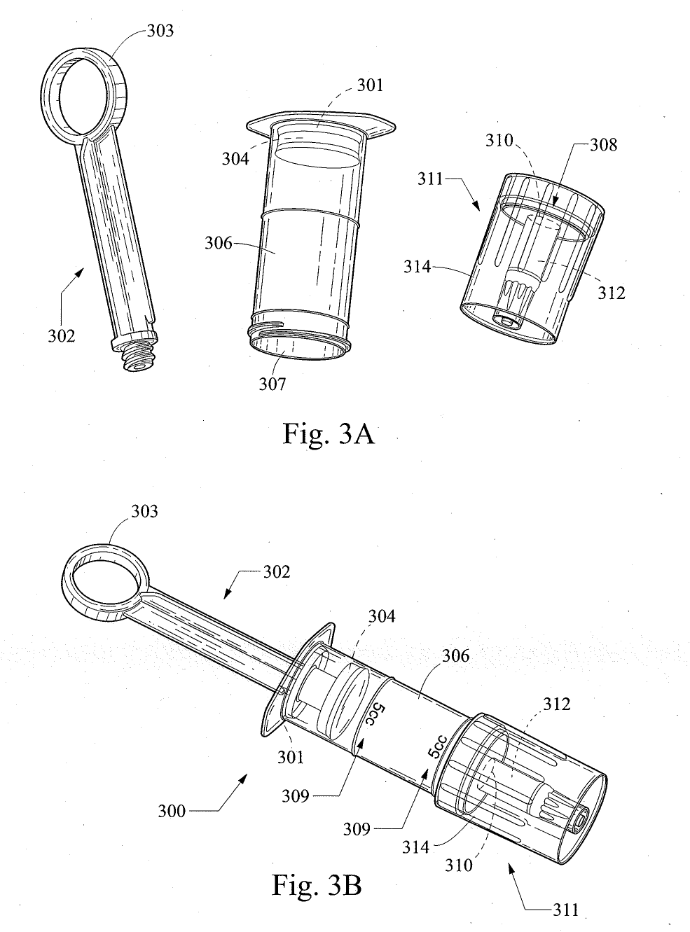

An embodiment of an instillation/aspiration assembly is described with reference to FIGS. 3A-3B, which respectively show a disassembled and an assembled instillation/aspiration assembly embodied as a syringe 300. The syringe 300 includes a removable handle 302 configured to be attached to/detached from a plunger 304 and a barrel 306, the interior portion of which includes a barrel lumen 307. The handle 302 preferably includes a thumb-ring portion 303 and is configured to actuate the plunger 304 distally and proximally along a central longitudinal axis of the barrel 306. The barrel 306 may be shaped and dimensioned as a standard sputum cup used for collection of bronchoalveolar lavage fluid samples. A proximal plunger-retention structure 301 preferably prevents the plunger 304 from being withdrawn completely through the proximal end of the barrel 306.

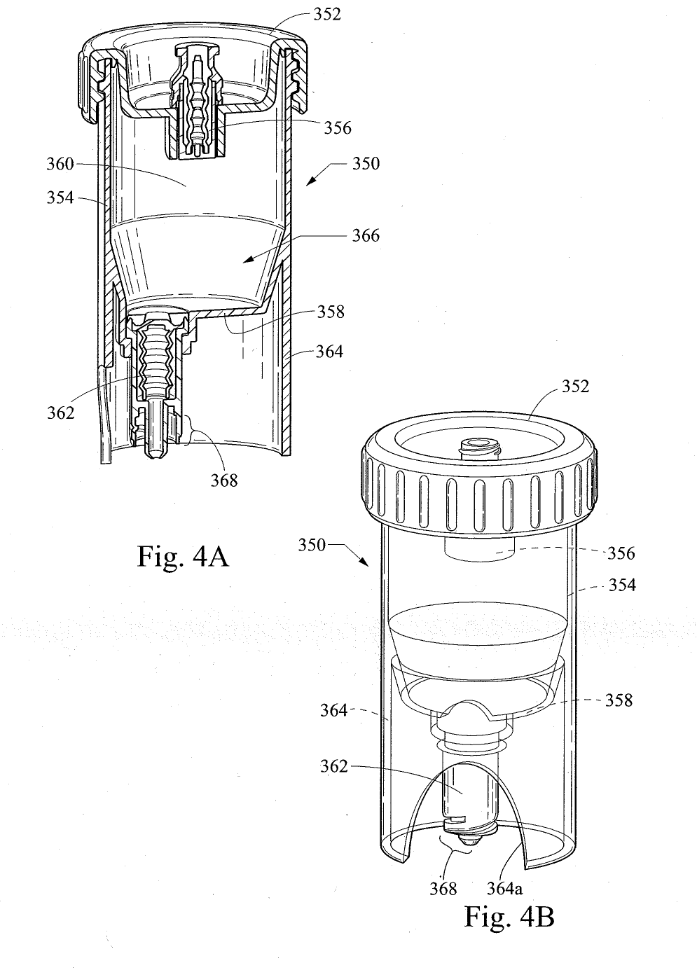

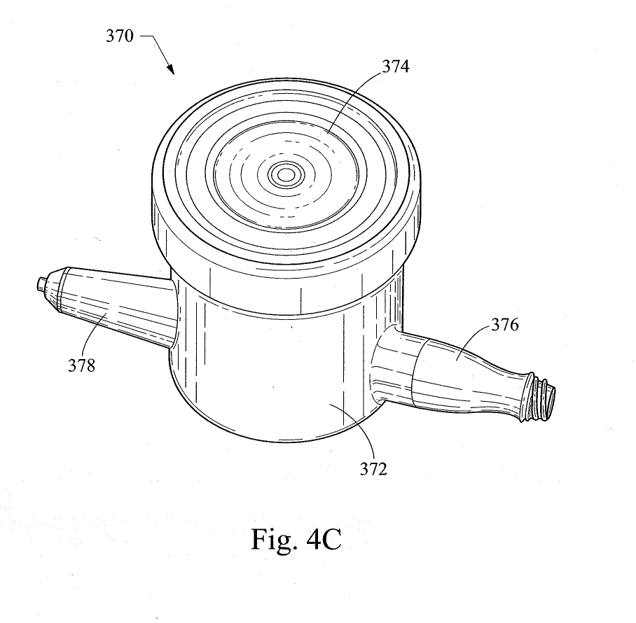

A cap member 311 is preferably configured for removable attachment to the distal end of the barrel 306. A proximal face of the cap member 311 is configured to form a distal wall 308 of the barrel lumen 307 when the cap member is attached to the barrel. In this embodiment, the proximal face of the distal wall 308 is generally planar and transverse to a longitudinal axis of an assembled syringe 300 and includes a central aperture 310 adjacent its distal end. In other embodiments (see, e.g., FIG. 4A), the wall 308 may be generally frustoconical in shape (truncated cone) or may otherwise be configured in a manner desired for efficient passage of fluid therethrough. The cap member 311 includes a self-sealing member 312, which extends distally from the aperture 310 and preferably is flush disposed or else recessed distally relative to the proximal surface of the wall 308.

The self-sealing member 312 preferably provides a fluid-disruptable seal to the aperture 310. In a preferred embodiment, the disruptable seal remains intact until the cap member is properly and completely connected with another device such as, for example, a catheter manifold. When that other device is removed, the seal is reinitiated. The cap member includes a generally cylindrical side wall portion 314 that extends distally beyond the wall 309 and circumferentially surrounds the self-sealing member 312.

Some or all of the barrel 306 and cap member 311 may be constructed of a transparent or translucent material to allow a user to view the contents of the barrel lumen. Also, a cut-out portion (not shown, see one example in FIG. 4B) may be provided in the side wall 314 of the cap member 311 to allow tactile access to the self-sealing member 312 by a user (e.g., to ease connection of the self-sealing member 312 to a catheter device). In one embodiment, the cap member 311 may be configured for a sealing threaded connection with the barrel 306, and the self-sealing member 312 may be configured for connection to a catheter device by a Luer-type or other fluid-patent connection.

In one embodiment, the barrel 306 may include a transparent or translucent portion allowing a user to see contents of the barrel and may also include one or more graduated volumetric indicia such as the notation “5 cc” (309) shown on the barrel exterior in FIG. 3B, which preferably indicates at least a minimum predetermined volume. In the illustrated embodiment, when the syringe 300 is assembled, the aperture 310 and self-sealing member 312 are disposed in line with its central longitudinal axis, but those of skill in the art will appreciate that, as is known with other syringes, the distal aperture 310 and structure extending distally therefrom may be disposed outside that central longitudinal axis. In certain commercial applications, it may be advantageous to preload the barrel lumen 307 with an aqueous solution such as, for example, a sterile saline solution, and provide the preloaded syringe to a user. In each of the embodiments presented herein, the self-contained nature of the instillation/aspiration assembly presents advantages over the prior art including that a collected fluid sample does not have to be transferred to another container before being transported to a laboratory for analysis, thereby lessening the likelihood of spillage. A significant advantage is that the presently-described device significantly reduces the likelihood of contamination of a collected sample because it has little or no exposure from the time it is collected in the lung until it undergoes analysis. This feature reduces the possibility of “false positives” and/or misidentification, during testing, of microbes actually infecting the patient that would result in unnecessary treatment of a patient, which—in turn—provides advantages of saving the patient on costs of treatment, saving the care providers' time and human resources, minimizing the patient's exposure to unneeded drugs, and accordingly lessening the unnecessary use of antibiotics associated with the dangerous increase of antibiotic-resistance in hospital microbes. It also reduces the likelihood of exposure of care-giving personnel to any microbes in the sample.

Other embodiments of an instillation/aspiration assembly are described with reference to FIGS. 4A-4C. FIGS. 4A-4B respectively show first and second embodiments of an instillation/aspiration assembly 350. The instillation/aspiration assembly 350 includes a lid member 352, and a barrel member 354. The lid member 352 includes a first self-sealing member 356, and a distal end wall 358 of the barrel lumen 360 includes a second self-sealing member 362. The outer barrel wall 364 extends distally beyond the second self-sealing member 362 and preferably includes a sufficiently regular surface to act as a base that will hold the instillation/aspiration assembly 350 upright on a flat surface. A distal portion of the barrel wall 364 includes a cut-out 364a that is configured to provide a user with tactile access for connecting, for example, a catheter device to the second self-sealing member 362. A distal surface region 366 of the barrel lumen 360 may be tapered inward to enhance efficient fluid flow to and through the second self-sealing member 362.

In the instillation/aspiration assembly 350 shown in cross-section in FIG. 4A, the second self-sealing member 362 is located off-center (i.e., out of a central longitudinal axis of the instillation/aspiration assembly 350), while, in the embodiment shown in FIG. 4B, the second self-sealing member 362 is generally centered (i.e., generally aligned along the central longitudinal axis of the instillation/aspiration assembly 350). The distal end 368 of the second self-sealing member 362 preferably is configured for engagement in a fluid-tight seal with a catheter device by, for example, a Luer-type or other threaded connection. The proximal end 370 of the first self-sealing member 356 preferably is configured for engagement in a fluid-tight seal with a syringe (not shown). An operation of the instillation/aspiration assembly 350 may include providing the barrel member 354, filling the barrel lumen 360 with, for example, a sterile aqueous solution and sealingly attaching the lid member 352 to the barrel 354. A syringe (not shown) may be provided with the plunger withdrawn and its body filled with a fluid such as, for example, air or a sterile aqueous solution. The syringe may be sealingly attached to the first self-sealing member 356. The second self-sealing member 362 may be attached to a catheter device (not shown) in fluid communication with an instillation/aspiration target site. The syringe may be distally-actuated to force the solution of the barrel lumen 360 out through the catheter to the target site and then proximally actuated to aspirate the solution back into the barrel lumen 360.

FIG. 4C is an alternative embodiment of the instillation/aspiration assembly shown in FIGS. 4A-4B. As shown in FIG. 4C, an in-line instillation/aspiration assembly 370 is provided. The instillation/aspiration assembly 370 includes a barrel body 372 housing a barrel body lumen, a removable cap 374, a proximal self-sealing member 376, and a distal self-sealing member 378.

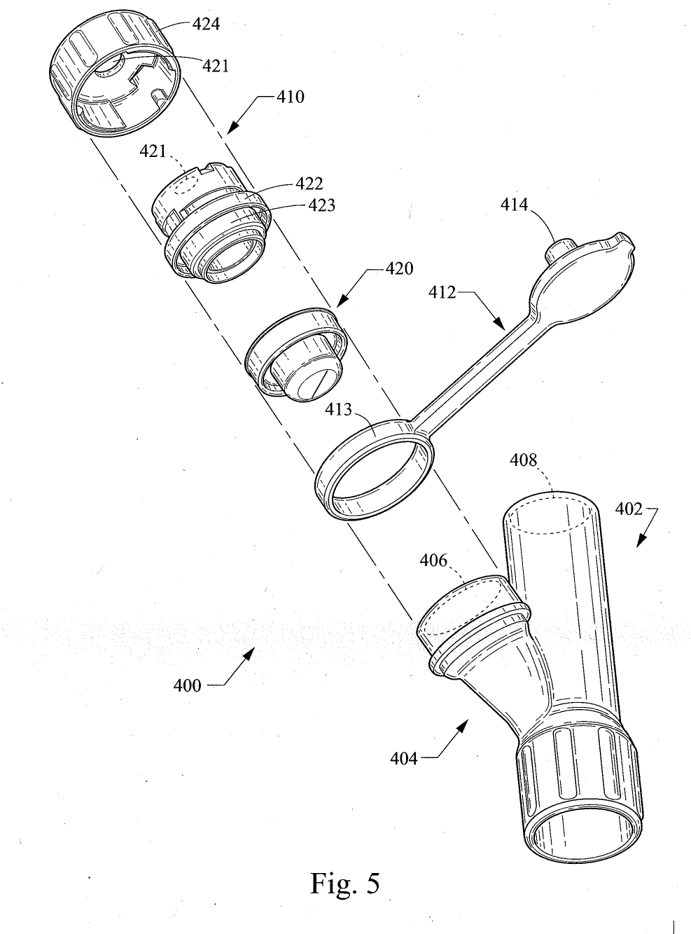

FIG. 5 shows an exploded view of the manifold assembly 400 depicted in FIG. 2. The manifold assembly 400 includes a generally tubular main body 402 with a generally tubular side branch 404 disposed at an angle to the main body 402. The side branch 404 includes a side branch lumen 406, which is continuous with a longitudinal main body lumen 408. A retaining ring portion 413 of a cap member 412 is configured to encircle a proximal exterior portion of the side branch 404, and a protrusion 414 of the cap 412 is configured to engage and generally seal a proximal central opening 421 of the cammed twist-lock mechanism 410. A dual-diaphragm seal 420 (described below with reference to FIGS. 7A-7C) is configured to be disposed in the side branch lumen 406. A cammed twist-lock mechanism 410 (described below in greater detail with reference to FIGS. 6A-6G), includes a central opening 421 configured for passage therethrough of a tubular body, such as a catheter. The cammed twist-lock mechanism 410 includes a hub member 422 and a knob member 424 configured to rotatably engage with the hub member 422. A distal portion 423 of the hub member preferably is configured to be attached into a proximal end portion of the side branch lumen 406.

The cammed twist lock mechanism is here described with reference to FIGS. 6A-6G. Distal and proximal perspective views, respectively, of the knob member 424 are shown in FIGS. 6A and 6B. The knob member 424 preferably includes ribs 440 on its outer circumference to promote grippability by a user. The inner circumferential surface of the knob member 424 includes a stop-ridge 442, which itself includes a detent tooth 442a. The inner circumferential surface also includes a rounded tracking-detent projection 444. A central circular opening 446 provided through the proximal knob wall 448 preferably is dimensioned to receive a tubular device such as, for example, a catheter. FIGS. 6C-6D show, respectively, distal end perspective and a side/proximal end perspective views of the hub member 422, and FIG. 6E shows a longitudinal section of the hub 422 along a line 6E-6E of FIG. 6C. The hub member 422 includes on its outer circumferential surface a detent-tooth-receiving track 450 and a stop-ridge-engagement projection 452. The outer circumferential wall surface of the hub 422 also includes a tracking-detent-receiving track 454 with a detent-capture bump 455 near one end. An off-center hub opening 456 is provided through the proximal hub wall 458.

The knob 424 is configured to engage the hub 422 such that the rounded tracking detent 444 of the knob 424 engages the corresponding track 454. Likewise the detent-tooth-receiving track 450 will engage the detent tooth 442a. As is described here with reference to FIGS. 6F-6G, the knob 424 is rotatable in a predeterminedly limited fashion relative to the hub 422. Specifically, when the knob 424 is rotated relative to the hub 422, the rounded detent 444 will ride along its track 454. At the same time, the detent tooth 442a will ride along its track 450. The knob 424 and hub 422 are dimensioned such that the rounded detent 444 will be captured by the detent-capture bump 454 at the same time the stop-ridge 442 and detent tooth 442a contact and are stopped from further rotary advancement by the stop-ridge-engagement projection 452. This final orientation provides a locked state shown in FIG. 6G.

As shown in FIG. 6F, when the cammed twist-lock mechanism 410 is in an unengaged/default position, the openings 446, 456 of the knob 424 and hub 422 are aligned (to form a central opening 421) such that a tubular member 460 may freely pass therethrough. When the knob 424 is rotated into the locked state, as shown in FIG. 6G, the knob opening 446 becomes misaligned from the off-center hub opening 456. The resulting interference frictionally captures the tubular member 460. Most preferably, the resulting interference results in a frictional binding of the tubular member 460 that does not significantly reduce its inner diameter, such that a second tubular member 461 disposed coaxially through the first tubular member 460 can still pass generally freely therethrough.

A dual-diaphragm seal 420 described here with reference to FIGS. 7A-7C is configured to be disposed in the side branch lumen 406 of the manifold 400. FIG. 7A shows a distal/side perspective view, FIG. 7B shows a proximal end perspective view, and FIG. 7C shows a section view along a line 7C-7C of FIG. 7A. A distal diaphragm 470 forms the distal wall of the seal 420 and includes at least one transverse slit 472 extending through its thickness. The seal 420 preferably is constructed of a resilient elastomeric material such that the slit 472 will, when closed, maintain a substantially fluid-tight seal. Preferably, the slit 472 is dimensioned and configured to allow passage of a tubular member such as, for example, a catheter (not shown) and to return to a self-sustaining seal when a tubular member or other intervening item is not present. A proximal diaphragm 474 includes a central opening 476 with a rounded margin 478 that is configured to maintain a substantially fluid-tight seal around the outer circumference of a tubular member such as, for example, a catheter (not shown) when such is passed through the seal. The dual-diaphragm construction of the seal 420 therefore provides for a substantially fluid-tight seal both in the presence and in the absence of a tubular member passing therethrough (including for example, the outer catheter of a catheter assembly of the present invention).

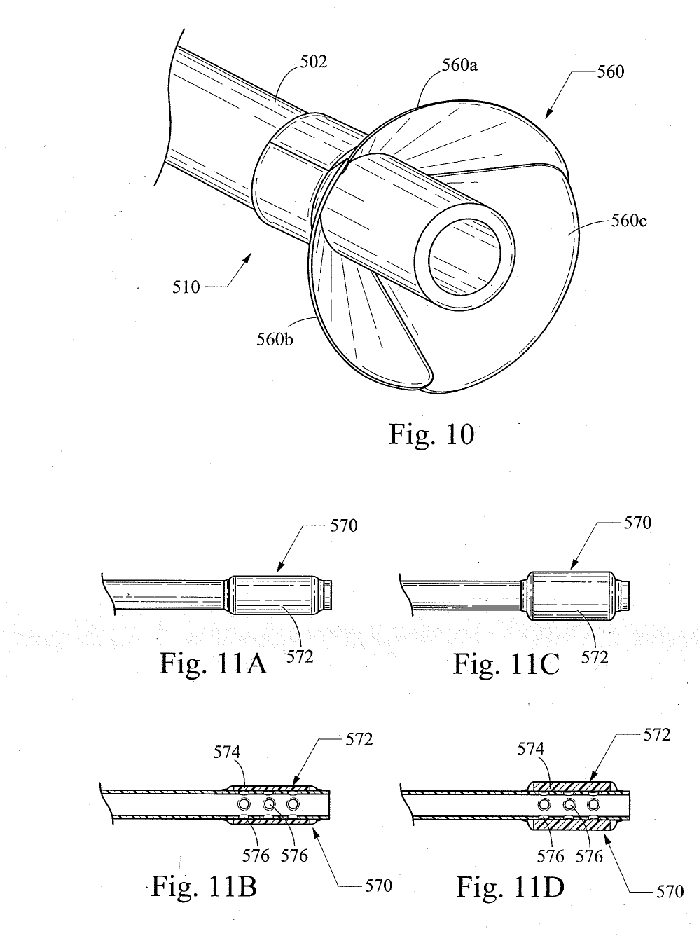

FIG. 8 shows an exploded view of a system assembly. An inner catheter 502 includes a proximal connection hub 504, preferably configured for substantially fluid-tight connection with an instillation/aspiration device in a manner providing a patent fluid path between that device and an inner catheter lumen 503 extending longitudinally through the length of the inner catheter 502. A distal end portion 510 of the inner catheter 502 includes a wedging structure 512, which is described in greater detail below with reference to FIG. 8A. Several other embodiments of wedging structures are described below with reference to FIGS. 10, 11A-11D, and 12A-12B. The inner catheter 502 preferably is dimensioned to be longitudinally coaxially passable through a lumen of the outer catheter 506.

The outer catheter lumen 507 (see FIG. 8B) extends longitudinally through substantially the entire length of the outer catheter 506. A squeeze-lock component 520, described below with reference to FIG. 8B, is attached to the proximal end of the outer catheter 506. An intermediate portion of the outer catheter 506 includes a preformed bend 514, as described above with reference to FIG. 2. The distal end 508 of the outer catheter 506 has an atraumatically-shaped tip 509 that includes a disruptable seal, which is described in greater detail below with reference to FIGS. 8C-8D. An outer sheath 530 is configured to attach proximally at a junction of the outer catheter 506 and the squeeze-lock component 520. The outer sheath 530 is configured to contain substantially the entire length of the outer catheter 506, and includes a removable distal portion 532 that is removable along a perforation or other separation means 534. One or more perforations or other separation means may be included near the distal end, at an intermediate location, and/or may be included near the proximal end (in the latter case, allowing removal of a larger portion including up to substantially the entire outer sheath).

FIG. 8A shows one embodiment of a wedging structure 512 on the distal end portion 510 of the inner catheter 502. The wedging structure 512 includes three flexible intact disc structures 512a-c that extend generally transversely (relative to the central longitudinal axis) around the outer circumference of the inner catheter 502. The discs 512a-c are shown as having the same outer diameter, but may have different outer diameters than each other. Also, although the discs are shown as being generally centered on the inner catheter 502, one or more of them may be mounted off-center. Preferably the wedging structure embodiment shown in FIG. 8A will include at least two discs, but it may include only one disc or more than the three discs illustrated, within the scope of the present invention. Additionally, the discs may be disposed about the inner catheter itself, or may be disposed on a separate end component that is affixed to the distal end of, and is substantially continuous with, the inner catheter. Such a separate end component may be affixed by, for example, adhesive, overmolding, or other attachment means.

One embodiment of a squeeze-lock component 520 is described with reference to FIG. 8B. A generally cylindrical grip portion 522 forms the proximal end portion of the squeeze-lock component 520. A body portion 524 extends distally from the grip portion 522. The grip portion 522 includes an obround lumen 526 disposed longitudinally therethrough and opposed ridged external grasp-surfaces 523. A pair of divots 528, which meet in an aperture 529, are disposed along opposite sides of the body portion 524 and provide enhanced flexibility for the grip portion 522. A generally cylindrical body lumen (not shown) extends longitudinally through the body portion 524 and is generally aligned and continuous with the obround grip lumen 526. When in a default state, the obround grip lumen is configured to grip the inner catheter 502 in a manner limiting, or—preferably—preventing its longitudinal movement. A user may distort the obround grip lumen 526 to release its frictional grip on the inner catheter 502 by exerting pressure on the opposed ridged external grasp-surfaces 523.

Another embodiment of a squeeze-lock component 800 is described with reference to FIGS. 8E and 8F. A grip portion 830 forms the proximal end portion of the squeeze-lock component 800. A generally cylindrical body portion 810 extends distally from the grip portion 830. The grip portion 830 includes lower and upper proximally-extending grip members 831, 832, which are biased apart by an intervening bias tab 839. The lower grip member 831 includes an upward-projecting grip-tab 833, which has an obround aperture 835 therethrough. The upper grip member 832 includes a downward-projecting grip-tab 834, which has a circular aperture 836 therethrough. The bias-tab 839 has a circular opening 841 therethrough that is aligned around the central longitudinal axis of the squeeze-lock component 800, as is a central generally columnar lumen 812 of the body portion 810. In some embodiments, it may be desirable to include an insert with a low-friction lumen (not shown) through some or all of the opening 841 and/or the body lumen 812 so provide for ease of a catheter's passage therethrough when not engaged with inner surfaces of the friction-locking apertures 835, 836.

One of skill in the art will appreciate the elegant simplicity for operation of this configuration. In an engaged configuration (i.e., substantially locking the inner catheter such that will not move longitudinally relative to the outer catheter through which it extends), the outward bias of the grip members 831, 832 will capture the inner catheter in grip-tab apertures 835, 836 and bias-tab opening 841. In a disengaged configuration (i.e., substantially allowing free longitudinal movement of the inner catheter relative to the outer catheter through which it extends), the grip members 831, 832 are squeezed together (against their bias) in a manner allowing free longitudinal movement of the inner catheter through their apertures 835, 836 as well as through the bias tab opening 841 and the body lumen 812. In certain embodiments, the proximal end of an outer catheter of the present invention will be attached to the distal squeeze-lock component body such that the outer catheter lumen is substantially continuous with the squeeze-lock component body lumen 812. It should be appreciated that use of a squeeze-lock component (of the type described herein, or another means for retaining the longitudinal position of the inner catheter relative to the outer catheter) in conjunction with the cammed twist-lock mechanism 410 described above provides a user with the ability to independently control longitudinal movement of one or both of the inner catheter 502 and the outer catheter 506.

FIGS. 8C and 8D show, respectively, side and side/end perspective views of the distal end 508 of the outer catheter 506. This distal outer catheter portion 508 includes a disruptable seal 509. The distal end portion 508 preferably includes an atraumatic shape, shown here as a domed tip. The disruptable seal preferably provides an effective barrier against microbes. In this manner, the outer catheter 506 can provide protection for the inner catheter 502 through the upper respiratory passages in order to minimize a risk that the inner catheter 502, including its distal end and/or wedging means will be contaminated with and carry into the lower lung materials from the upper airway. When the outer catheter 506 is in a desired position, the seal 509 can be disrupted by pushing the inner catheter 502 out through it. The seal 509 is shown here as including two generally perpendicular slits 509a, 509b that extend from the outer catheter lumen 507 partially though the wall of the outer catheter 506, but other embodiments may have fewer or more slits. The position of the slits 509a-b forms four leaflets 509w-z that may be separated when the seal is disrupted (as is shown with reference to FIG. 2). One method of forming the disruptable seal 509 shown in FIGS. 8C and 8D is to begin with a polymer catheter having an open tubular end, use a heated mold to form the end into a completely sealed dome tip, incise a pair of crossed slits to create leaflets therein, then to re-heat the slit domed tip in a manner creating a membrane-type seal across only a thickness portion of the slits (i.e., the entire thickness of each slit is not reconnected, but only an outer thickness portion).

Another embodiment of a disruptable seal for an outer catheter is described with reference to FIGS. 9A-9E. FIGS. 9A and 9B illustrate, respectively, an external and a partial section view of a disruptable seal 550. The seal 550 is shown as a separate component from the outer catheter 506, but could be seamlessly integrated with it. The seal 550 includes three leaflets 550a-c that, in a closed configuration are sealed together in a manner preferably resisting or preventing passage therethrough of microbes. As shown in FIG. 9C, the internal surface of each of the leaflets 550a, 550b, 550c includes a curved camming surface 550x, 550y, 550z (respectively). As in the embodiments described above, the inner catheter 502 is disposed coaxially through the lumen of the outer catheter 506. FIGS. 9D-9E show a method of use for opening the disruptable seal 550. In order to disrupt the seal 550 and extend the inner catheter 502 (shown here without wedging means for the sake of illustrative clarity) beyond the distal outer catheter end 508, the inner catheter 502 is pushed distally to exert force against the camming surfaces 550x-z. This force cams open the leaflets 550a-c, permitting the inner catheter 502 to exit distally.

Another embodiment for a wedging tip for an inner catheter is described with reference to FIG. 10. In this embodiment, the distal end portion 510 of the inner catheter 502 includes an “Elizabethan collar” wedging structure 560. The generally frustoconical collar wedging structure 560 includes three leaflets 560a-c formed of a flexible material. The leaflets 560a-c are biased into the open configuration shown in FIG. 10, but preferably are configured to collapsibly overlap each other in a manner allowing them to have a collapsed outer diameter permitting the inner catheter 502 to pass freely through the outer catheter lumen. Then, when the inner catheter 502 is extended distally through and beyond a disruptable seal of the outer catheter 506, the leaflets 560a-c will assume their default/biased position. This expanded configuration preferably is dimensioned to circumferentially contact the walls of a bronchial passage as is known in the art for the purpose of wedging during a bronchoalveolar lavage procedure. Those of skill in the art will appreciate that other embodiments not requiring multiple leaflets, but instead including a single expandable collar member (e.g., with an elastically expanding, accordion-style, or other expanding means being used) may also be practiced within the scope of the present invention.

FIGS. 11A-11D illustrate another embodiment of a wedging structure for an inner catheter. A swellable wedging structure 570 is provided near the distal end 510 of an inner catheter 502. FIGS. 11A and 11B show, respectively, an external side view and a longitudinal section view of the wedging structure 570 in its low-profile/unexpanded state. The wedging structure 570 includes an outer balloon member 572 and a swellable absorbent material 574 between the balloon 572 and the outer wall of the inner catheter 502. The material 574 may include, for example, an absorbent polymer that expands in the presence of an aqueous solution. The portion of the inner catheter 502 immediately adjacent and surrounded by the balloon 572 includes a plurality of apertures 576 providing a path of fluid communication between the inner catheter lumen 503 and the swellable material 574. FIGS. 11C and 11D show, respectively, an external side view and a longitudinal section view of the wedging structure 570 in its high-profile/expanded state. The expanded state may be effected by introduction of an aqueous solution through the inner catheter lumen 503 such that the solution can pass through the apertures 576 into the swellable material 574, which is expanded thereby.

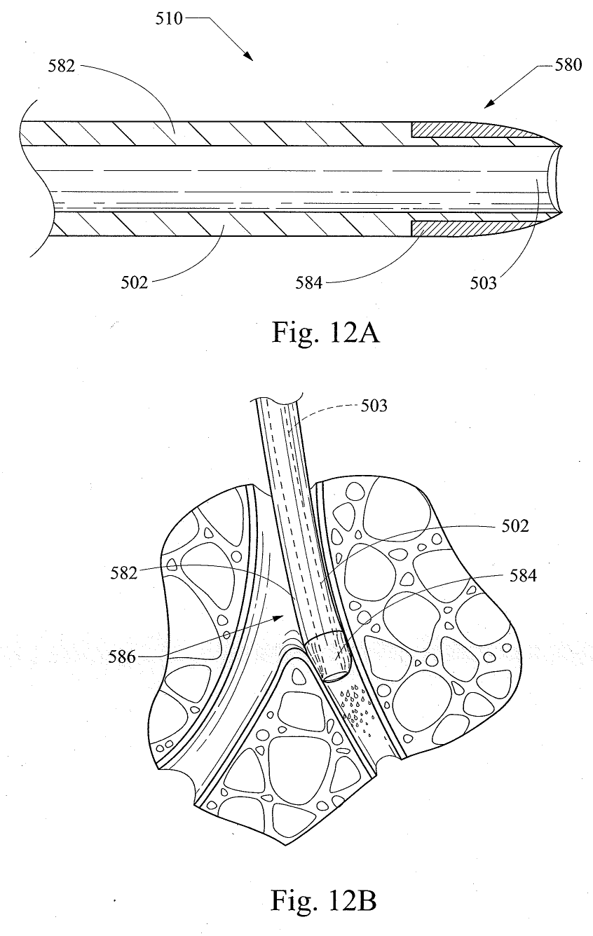

Another wedging structure embodiment is described here with reference to FIGS. 12A-12B. A molding-tip wedging structure 580 is provided on the distal end region 510 of an inner catheter 502. FIG. 12A shows the molding-tip wedging structure 580 in longitudinal section. The inner catheter 502 includes a first flexible material 582 suitable for use as a catheter body, and having limited radial compressibility. The molding-tip wedging structure 580 comprises a second flexible material 584 that is radially compressible/moldable. As shown in FIG. 12B, the molding-tip wedging structure 580 can be directed into a substantially circumferentially sealing contact with the inner circumference of a bronchial passage 586. The moldability of the second material 584 provides for enhancement of the wedging seal, while the first material 582 preferably retains the patency of the inner catheter lumen 503.

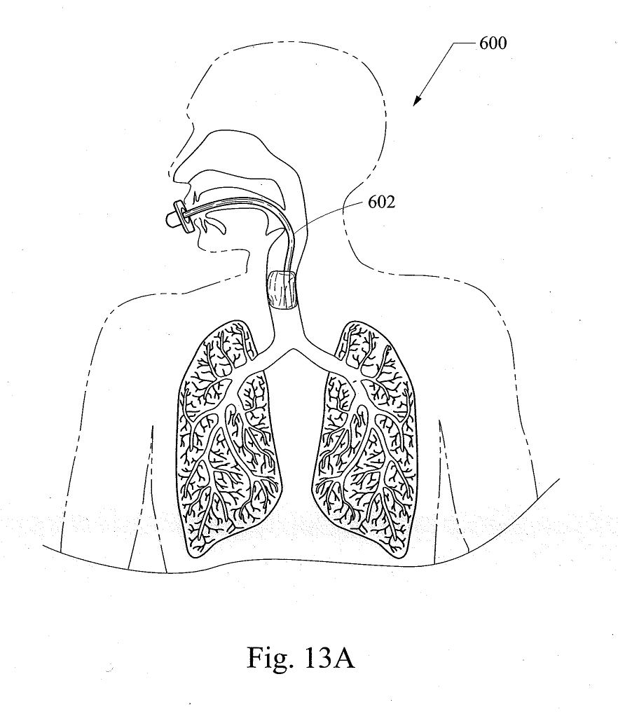

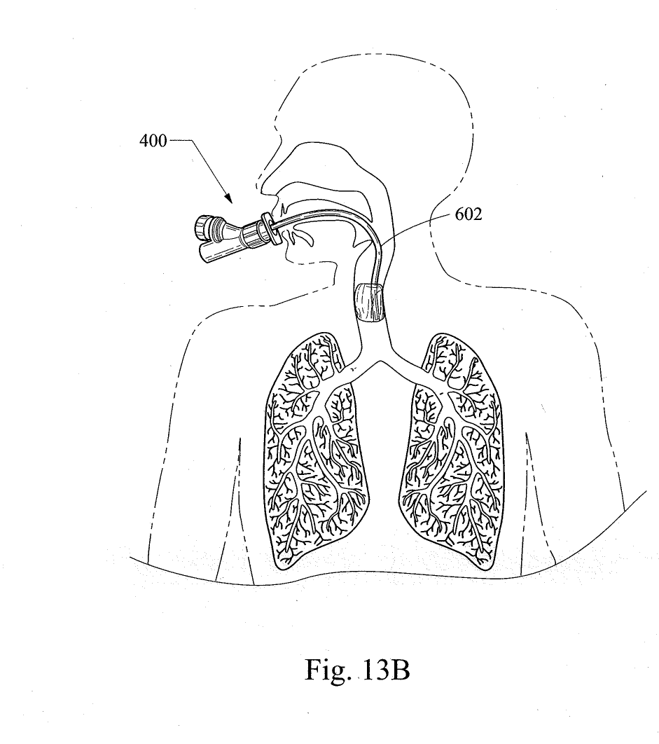

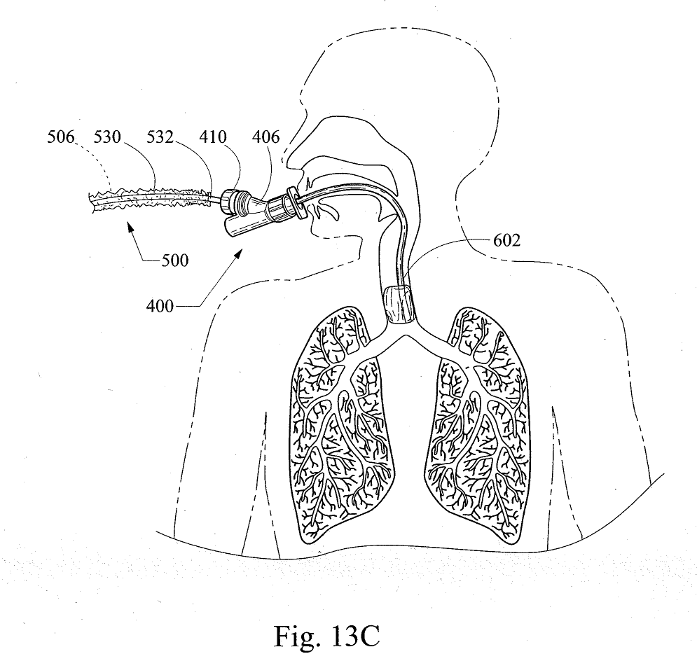

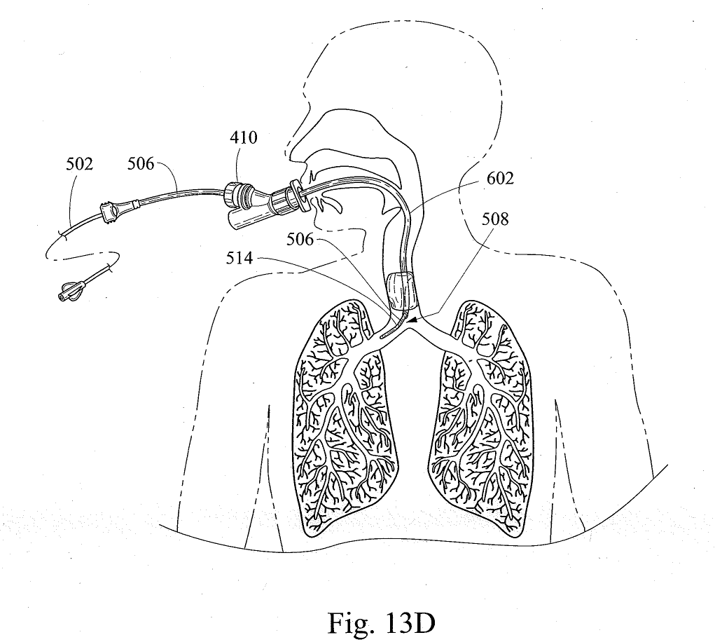

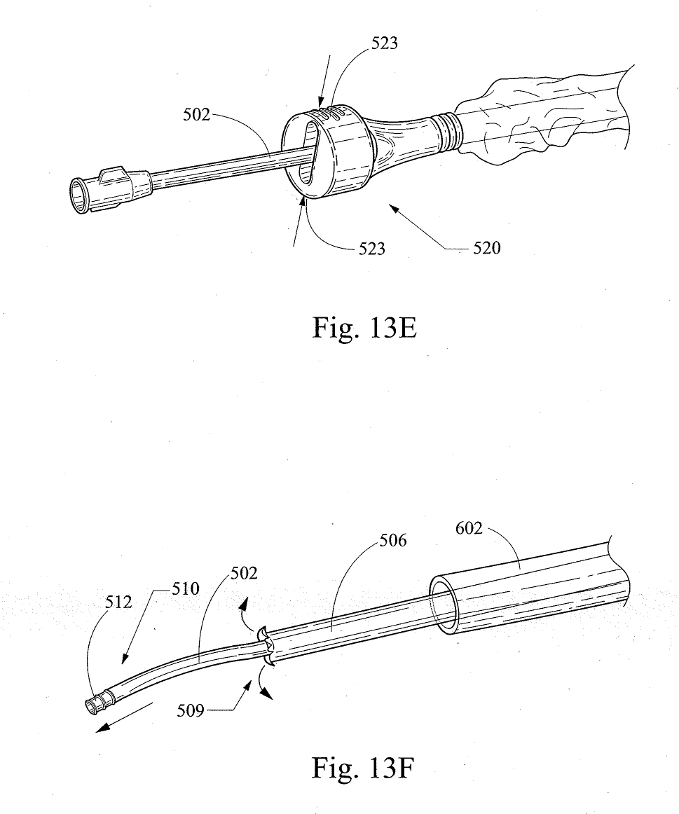

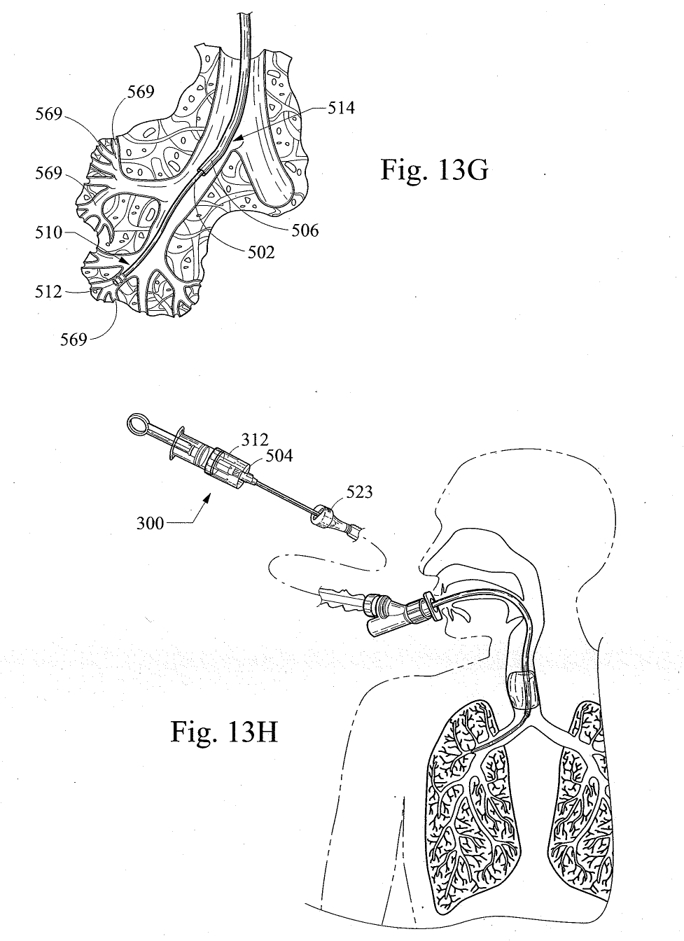

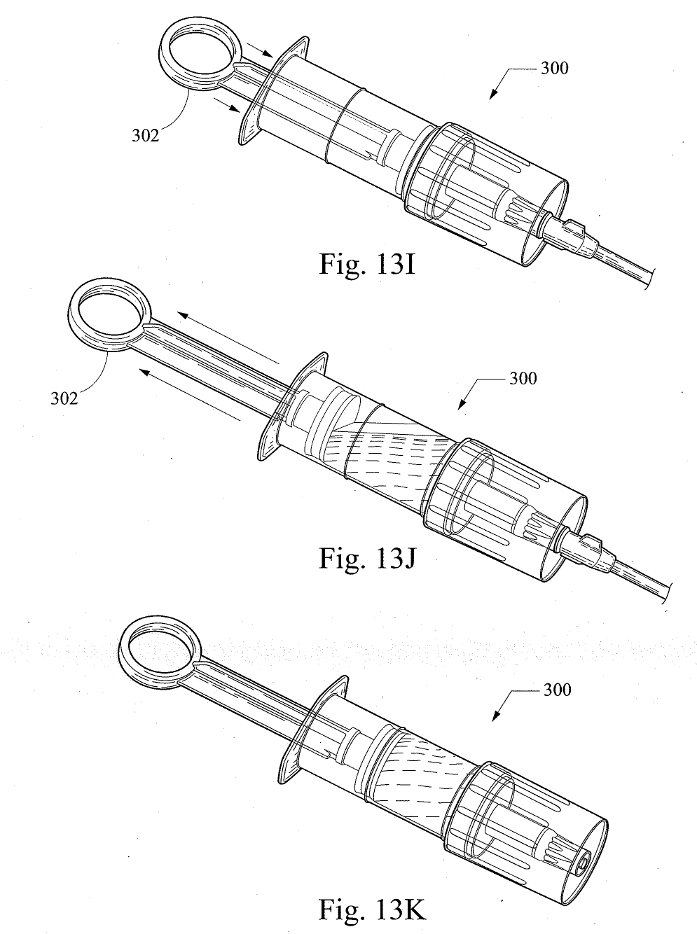

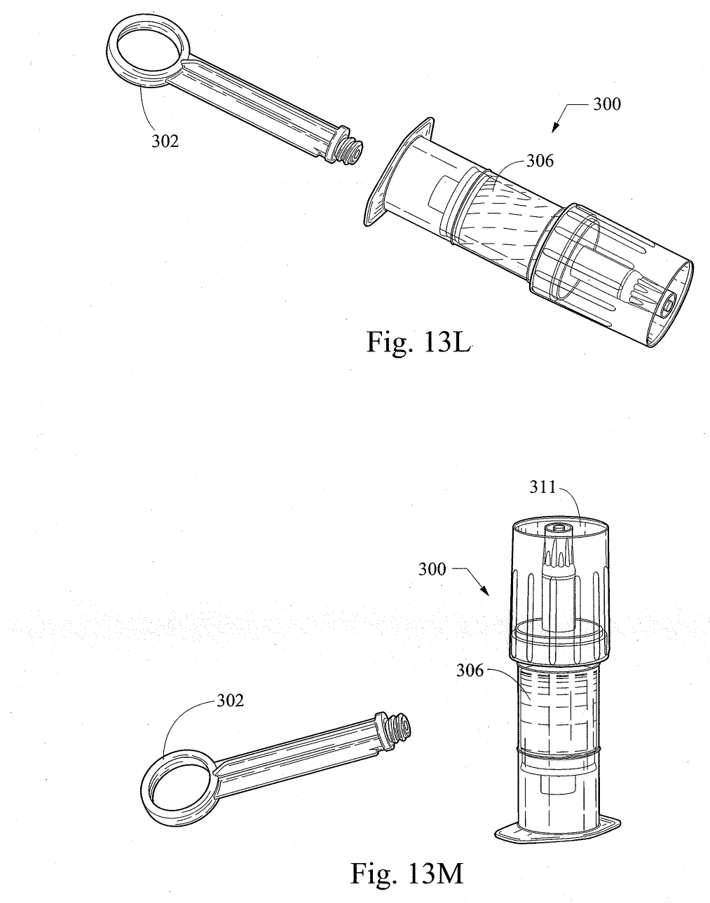

A method of using a bronchoalveolar lavage system of the present invention is described with reference to FIGS. 13A-13M. As shown in FIG. 13A, a patient 600 is provided with an endotracheal tube 602 (as used with reference to various aspects of the present invention, the term “endotracheal tube” is used generically to include a traditional/transpharyngeal endotracheal tube, a tracheostomy tube, and any currently-known or future-developed variants thereof). Next, as shown in FIG. 13B, a manifold assembly 400 is attached to the endotracheal tube 602. In a patient treatment setting, a manifold configured for use with a system of the present invention may already be provided in a patient's set-up. A catheter assembly 500, as described above with reference to FIGS. 2 and 8-8D, is provided and the distal portion 532 of the outer sheath 530 is opened or removed to allow passage of the outer catheter 506, which contains the distal length of the inner catheter 502 (not shown). The twist-lock mechanism 410 on the manifold side branch 406 is placed in an open/unlocked state, and the outer catheter 506 is directed through it as shown in FIG. 13C. As depicted in FIG. 13D, the outer catheter 506 is advanced distally through the endotracheal tube 602 until its distal end portion 508 exits in the lower trachea and the bend 514 is gently oriented to direct the distal end 508 of the outer catheter 506 toward the desired lung. The step illustrated with reference to FIG. 13D may be more easily effected by placement of visual indicia on a proximal portion of the outer catheter 506. Specifically, the outer catheter 506 may include first visual indicia 590 in the form of a graduated marking showing the distance (e.g., in inches or centimeters) from the distal end of the outer catheter 506. It may also include second visual indicia 592 on one radial portion that indicates which direction the distal region of the outer catheter 506 is curved or bent, such that the outer catheter can be directed into the endotracheal tube 602 at an initial orientation consistent with placing the outer catheter into the desired (left or right) lung upon its exit from the distal end of the tube 602. A standard endotracheal tube 602 commonly includes visual indicia (not shown) in the form of partial or complete bands at 26 cm and 28 cm from the distal end of that tube 602. During a standard placement procedure, advancing the distal end of the outer catheter about 5 cm beyond the distal end of that tube 602 will clear the carina and place the outer catheter 506 in a position where it is desirable to deploy the inner catheter 502. The user may then actuate the twist-lock mechanism 410 to prevent further longitudinal movement of the outer catheter 506.

Next, as indicated in FIG. 13E, the user may actuate the squeeze-lock component 520 by pressing its opposed ridged external grasp-surfaces 523 together to release its frictional grip on the inner catheter 502. Then, as shown in FIG. 13F, the user may distally advance the inner catheter 502 in a manner disrupting the distal seal 509 of the outer catheter 506. As illustrated in FIG. 13G, the user may advance the inner catheter 502 until the wedging structure 512 of its distal end 510 is sealed into a bronchial passage 569 of the patient. This may be done “by touch,” which may be helped by graduated visual indicia (not shown) on the inner catheter 502. After releasing the opposed ridged external grasp-surfaces 523 of the squeeze-lock component 520 to longitudinally hold the inner catheter 502 in place, the user may connect the self-sealing member 312 of an instillation/aspiration device 300, preferably preloaded with a sterile saline solution, to a hub 504 of the inner catheter as is shown in FIG. 13H.

The user then may instill (FIG. 13I) and aspirate (FIG. 13J) the saline solution by, respectively, distally advancing then proximally retracting the handle 302. After the solution is collected as a fluid sample, the instillation/aspiration device 300 may be removed from the catheter assembly 500 (FIG. 13K, with reference to FIGS. 2 and 13H), the handle 302 may be removed (FIG. 13L), and the remaining portion of the instillation/aspiration device 300 (FIG. 13M) is ready for use in transporting the fluid sample to a site for analysis. Thereafter, a technician desiring to perform an analysis may remove the cap member 311 for access to the fluid sample (see FIG. 3A). Those of skill in the art will appreciate that other embodiments of components that are described herein and/or are developed in the future may be used in accordance with this method within the scope of the present invention.

Those of skill in the art will also appreciate that different embodiments of components described in the present application, known in the art, and/or developed in the future may be used as part of assemblies and systems described and claimed herein within the scope of the present invention. It is therefore intended that the foregoing detailed description be regarded as illustrative rather than limiting, and that it be understood that it is the following claims, including all equivalents, that are intended to define the spirit and scope of this invention.

We claim:

1. A bronchoalveolar lavage instillation/aspiration system comprising:
a needleless bronchoalveolar lavage instillation/aspiration device comprising a barrel member including a barrel lumen where a distal portion of the barrel member further comprises
a distal aperture between the barrel lumen and an exterior region distal of the barrel member and
a self-sealing member configured for maintaining a fluid-disruptable, resealable barrier to the distal aperture; and
a catheter assembly configured to provide a patent path of fluid communication between the instillation/aspiration device and a patient bronchial passage, the catheter comprising a wedging structure configured to engage a patient bronchial passage.

2. The system of claim 1, where the instillation/aspiration device further comprises:
a plunger member disposed slidably in the barrel lumen and providing a generally proximal seal for the lumen; and
a handle member attached to, and configured to axially actuate, the plunger member, where the handle member is removable from the plunger member.

3. The system of claim 1, where the instillation/aspiration device further comprises:
a proximal aperture between the barrel lumen and an exterior region proximal of the barrel member including a second self-sealing member configured for maintaining a fluid-disruptable, resealable barrier to the proximal aperture; and
an external proximal connection structure configured for forming a fluid-patent connection with a syringe.

4. The system of claim 3, further comprising a needleless syringe.

5. The system of claim 1, where a portion of the barrel comprises a removable cap member configured to provide unsealed external access to the barrel lumen.

6. The system of claim 1, where the distal portion of the instillation/aspiration device and a proximal portion of the catheter assembly are configured as mating Luer-type connections.

7. The system of claim 1, where the catheter assembly further comprises an inner catheter member having an inner catheter lumen and an outer catheter member having an outer catheter lumen, where the inner catheter member disposed is longitudinally and coaxially through at least a lengthwise portion of the outer catheter lumen, the inner catheter member having a greater length than the outer catheter member, a distal end portion of the outer catheter further comprising an atraumatically-shaped disruptable seal, the seal including at least one slit that extends at least partially through an internal distal end wall portion of the outer catheter, and a distal end portion of the inner catheter further comprising the wedging structure, said wedging structure configured to at least partially sealingly contact an inner circumference of a passage in a lower portion of a patient lung;
where the distal portion of the instillation/aspiration device and a proximal portion of the inner catheter are configured for connection together in a manner providing for a patent path of fluid communication between the barrel lumen and the inner catheter lumen.

8. The system of claim 7, where the wedging structure comprises at least one adjacent flexible intact disc structure disposed around an outer circumference of the inner catheter and generally transverse to a longitudinal axis of the inner catheter.

9. The system of claim 7, where the atraumatically-shaped disruptable seal is configured to be disrupted by a distalward force exerted against an inner surface of the seal by the distal end portion of the inner catheter.

10. The system of claim 7, where the atraumatically-shaped disruptable seal is configured to provide an effective barrier against microbes between the inner catheter and an exterior region adjacent the distal outer catheter.

11. The system of claim 7, where the outer catheter comprises at least one lengthwise intermediate portion constructed of material sufficiently rigid to maintain a preformed bend.

12. The system of claim 11, where the at least one lengthwise intermediate portion comprises a preformed bend out of a generally linear default longitudinal axis, the preformed bend configured at a predetermined angle falling within a typical angle range of a patient's tracheal-bronchial junction.

13. The system of claim 11, where the preformed bend is about 30 degrees.

14. The system of claim 7, further comprising an elongate outer polymer sheath disposed about and containing substantially a majority length of the inner and outer catheter members, the polymer sheath configured to maintain a generally sterile state of that majority length.

15. The system of claim 14, where the outer polymer sheath comprises a removable portion configured for separation of the removable portion from the catheter assembly.

16. The system of claim 7, where the wedging structure comprises at least one flexible intact disc structure disposed around an outer circumference of a tip member attached to the inner catheter and generally transverse to a longitudinal axis of the inner catheter.

17. The system of claim 7, further comprising a manifold member that is configured for attachment to an endotracheal tube, the manifold member comprising a generally tubular main body and a generally tubular side branch at an angle to the main body, where a side branch lumen is continuous with a longitudinal main body lumen and is configured to provide passage for the outer catheter of the catheter assembly.

18. The system of claim 17, the side branch lumen further comprising a sealing diaphragm and a means for frictionally holding the outer catheter while allowing free passage of the inner catheter.

19. The system of claim 18, where the sealing diaphragm comprises:
a first membrane generally transverse to a central axis of the side branch where the membrane forms a sealing plane with a generally central slit therethrough, the slit being dimensioned to allow passage of the outer catheter, and
a second membrane adjacent and generally parallel with the first membrane, the second membrane including a generally central opening dimensioned to fit sealingly around an outer circumference of the outer catheter.

20. A method of performing a lavage procedure comprising the steps of:
providing the system of claim 1
providing a volume of an aqueous solution in the barrel lumen;
directing a distal end of the catheter assembly through a patient trachea into an upper bronchial passage;
holding the outer catheter member generally in place and directing the inner catheter member distally against the internal distal end wall portion of the outer catheter in a manner that disrupts the disruptable seal;
directing the inner catheter member into a lower bronchial passage until the wedging member substantially circumferentially contacts a wall of the lower bronchial passage;
connecting the instillation/aspiration device with the catheter assembly in a manner providing a patent path of fluid communication between the inner catheter lumen and the barrel lumen through the self-sealing member;
installing at least a predetermined portion of the aqueous solution to the site; and
thereafter aspirating at least a fraction of the predetermined portion of aqueous solution back into the barrel lumen.

21. The method of claim 20, where the instillation/aspiration device further comprises a plunger member disposed slidably in the barrel lumen and providing a generally proximal seal for the lumen and a handle member attached to and configured to axially actuate the plunger member, and where the step of installing further comprises actuating the handle member distally to direct the plunger member distally in the barrel lumen.

22. The method of claim 21, where the step of aspirating further comprises actuating the handle and plunger members proximally to create a partial vacuum in the barrel lumen.

23. The method of claim 20, where the handle member is removable from the plunger member and further comprising a step of removing the handle member.

24. A bronchoalveolar lavage instillation/aspiration system comprising:
a bronchoalveolar lavage instillation/aspiration syringe portion comprising:
a barrel member including a barrel lumen where a distal portion of the barrel member further comprises
a distal aperture between the barrel lumen and an exterior region distal of the barrel member and
a self-sealing member configured for maintaining a fluid-disruptable, resealable barrier to the distal aperture
a plunger member disposed slidably in the barrel lumen and providing a generally proximal seal for the lumen; and
a handle member attached to, and configured to axially actuate, the plunger member, where the handle member is removable from the plunger member; and
a catheter assembly comprising an inner catheter member having an inner catheter lumen and an outer catheter member having an outer catheter lumen, where the inner catheter member disposed is longitudinally and coaxially through at least a lengthwise portion of the outer catheter lumen, the inner catheter member having a greater length than the outer catheter member, a distal end portion of the outer catheter further comprising an atraumatically-shaped disruptable seal, the seal including at least one slit that extends at least partially through an internal distal end wall portion of the outer catheter, and a distal end portion of the inner catheter further comprising a wedging structure configured to at least partially sealingly contact an inner circumference of a passage in a lower portion of a patient lung;
where the distal portion of the instillation/aspiration device and a proximal portion of the inner catheter are configured for connection together in a manner providing for a patent path of fluid communication between the barrel lumen and the inner catheter lumen.

25. The system of claim 24, where the wedging structure comprises at least one adjacent flexible intact disc structure generally transverse to a longitudinal axis of the inner catheter and disposed around an outer circumference of the inner catheter.

26. The system of claim 25, where the disc structures are dimensioned to circumferentially contact a lower bronchial passage of a patient.

27. The system of claim 24, where the atraumatically-shaped disruptable seal is configured to be disrupted by a force exerted between an inner surface of the seal and the distal end portion of the inner catheter.

28. The system of claim 24, where the wedging structure comprises at least one adjacent flexible intact disc structure disposed around an outer circumference of a tip member attached to the inner catheter and generally transverse to a longitudinal axis of the inner catheter.

29. The system of claim 28, where the disc structures are dimensioned to circumferentially contact a lower bronchial passage of a patient.

30. The catheter assembly of claim 24, where the atraumatically-shaped disruptable seal is formed by sealing a thickness portion along the entire length of the slit.

31. A method of performing non-bronchoscopic bronchoalveolar lavage comprising the steps of:
providing an instillation/aspiration device comprising a self-sealing member and configured to introduce a fluid and collect, then transport at least a portion of the fluid;
providing a substantially sterile aqueous solution in the instillation/aspiration device;
providing a catheter assembly in patent fluid communication with the instillation/aspiration device;
directing a distal portion of the catheter assembly into a patient lung;
instilling into the patient lung and then aspirating back at least a portion of the solution;
disconnecting the instillation/aspiration device from the catheter assembly; and
transporting the aspirated solution in the instillation/aspiration device to a diagnostic facility.
